(19) United States
(12) Patent Application Publication
(10) Pub. No.: US 2004/0081646 A1
(43) Pub. Date: Apr. 29, 2004

(54) Group B Streptococcal phage lysin

(76) Inventors: Jeffrey A. Engler, Birmingham, AL (US); Jane R. Schwebke, Pell City, AL (US); John R. Baker, Birmingham, AL (US); David G. Pritchard, Indian Springs Village, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/650,559

(22) Filed: Aug. 28, 2003

Related U.S. Application Data
(60) Provisional application No. 60/407,038, filed on Aug. 29, 2002.

(51) Int. Cl. A61K 38/48; C12N 9/48

(52) U.S. Cl. 424/94.63; 435/212

(57) ABSTRACT

The present disclosure relates to the identification phage lysin from group B streptococci (GBS). The nucleic acid sequence and amino acid sequence of the GBS phage lysin is disclosed. The GBS phage lysin is a bifunctional protein comprising a glucosidase and an endopeptidease activity. The endopeptidase present in the GBS phage lysis has a substrate specificity that has not been previously reported. The bifunctional GBS phage lysin is effective against groups A, B, C, E and G streptococci. Also described are methods of using the GBS phage lysin for the prevention and treatment of bacterial infections.

### FIELD OF THE DISCLOSURE

The present disclosure claims priority to and the benefit of U.S. provisional application No. 60/407,038, filed Aug. 29, 2002. The research related to this disclosure was supported in part by a grant from the Public Health Service (Ai054897); the United States government may have some rights in this disclosure. The present disclosure relates to the identification of genes and proteins from bacteriophages of group B streptococci (GBS).

### BACKGROUND

Group B streptococci (GBS) are a major cause of serious and potentially fatal neonatal bacterial infections in this country (Centers for Disease Control and Prevention. 1996, Morbid. Mortal. Weekly Rev. 45(No. RR-7): 1-24.) GBS is also a common cause of serious infections in immunocompromised adults (Zangwill, et al. 1992. Morbid. Mortal. Weekly Rev. 41:35-32.). GBS phages were first isolated from bovine strains of GBS in 1969 and shown to be double-stranded DNA phages having relatively small heads and long tails (Russell, et al. 1969. J. Gen. Virol. 5:315-7). GBS phages were subsequently reported to be ubiquitous in human GBS strains and they formed the basis for various phage-typing systems for GBS (Haug, et al. 1981. J. Med. Microbiol. 14:479-82., Stringer, J. 1980. J. Med. Microbiol. 13:133-43). All double-stranded DNA bacteriophages that have been studied utilize a two-component lytic system which includes both a holin and a lysin. The holin disrupts the cell membrane thereby exposing the peptidoglycan to the degradative action of the lysin (Grundling, et al. 2001. Proc. Natl. Acad. Sci. U S A 98: 9348-52). There have been no reports describing the lysins of GBS phages.

Phage lysins typically possess two different functional domains, an N-terminal domain containing the catalytic site and a C-terminal domain that confers specificity (Sheehan, et al. 1996. FEMS Microbiol. Lett. 140:23-8.). In some cases the N-terminal domain of a phage lysin contains two different enzyme activities. For example, Navarre et al. (Navarre, et al. 1999. J. Biol. Chem. 274:15847-56) found that staphylococcal phage phill lysin possessed both a D-alanyl-glycyl endopeptidase, which cleaved linkages to glycine cross-bridges, and an N-acetylmuramidase acetylmuramidase (Navarre, et al., 1999). Chimeric lysins have even been constructed by joining different catalytic and peptidoglycan-binding regions of lysins (Sheehan, et al., 1996.).

### BRIEF DESCRIPTION OF THE FIGURES

FIG. 1 shows the susceptibility of various Streptococcus species to affinity-purified GBS phage lysin. The assay illustrates % of cellular lysis after two minutes of treatment with GBS phage lysin. Each strain was grown to an OD₅₅₀ of 0.4 in Todd Hewitt broth (mid-log growth phase) and concentrated by centrifugation. The cells were resuspended in lysin buffer A to an OD₅₅₀ of 0.8 and treated for two minutes in the presence of GBS phage lysin. Reduction in turbidity was then determined as described below.

FIG. 2A shows the reduction of bacterial turbidity of strain 3331 GBS cells by affinity-purified GBS phage lysin enzyme as a function of the growth rate of the substrate bacteria. To determine sensitivity to GBS phage lysin as a function of growth stage, samples were taken at various times after inoculation of the culture and the bacteria collected by centrifugation, washed with lysin assay buffer and resuspended in lysin assay buffer and the concentration adjusted so as to give an OD₅₅₀ of 0.8. A fixed amount of GBS phage lysin was added and the decrease in OD₅₅₀ measured as a function of time.

FIG. 2B shows the growth curve (as a function of time) of strain 3331 GBS cells from which the samples in FIG. 2A were taken.

FIG. 3 shows the homology alignment of lysins from bacteriophages that infect Streptococcus. Alignment of the sequences was performed by creating a multiple sequence alignment using a simplification of the progressive alignment method of Feng and Doolittle (Journal of Molecular Evolution 25; 351-360 (1987)). The group C phage lysin sequence was determined from the DNA sequence of a bacteriophage from S. equi, strain CF32 (GENBANK accession number AF186180). The group A phage lysin was determined from the DNA sequence of a putative bacteriophage lysin from S. pyogenes M1 GAS, strain SF370 (GENBANK accession number AE006546). The group B phage lysin was determined from the DNA sequence of a bacteriophage from a mitomycin-treated culture of GBS serotype III strain 3330 (GBS phage lysin, SEQ ID #1).

FIG. 4 shows agarose gel electrophoresis of GBS phage lysin PCR products. Lane 1 shows the product of the PCR reaction described below. Lane 2 shows size markers (1 kb ladder, Gibco-BRL). From the bottom, the markers are (506+517), 1018, 1636, 2036, 3054, 4072, then unresolved markers up to 12, 216 bp. The approximate size of the PCR product is 1300 bp.

FIG. 5 shows SDS polyacrylamide gel analysis of recombinant GBS phage lysin protein expression. Protein expressed in E coli BL21(DE3) cells was purified over a Ni-NTA affinity column as described below and aliquots of the various fractions separated by size by gel electrophoresis on a 8% SDS-polyacrylamide gel. Lanes (starting at left side of gel image) are: lane 1, supernatant applied to the Ni-NTA column (4 μl); lane 2, flow-through from column (4 μl); lane 3, wash 1 (4 μl); lane 4, elute 1 (3 μg loaded); lane 5, elute 2 (2 μg loaded); and lane 6: elute 3 (3 μg loaded). Because the column was overloaded with protein product, some of the protein was lost during initial wash steps.

FIG. 6 shows the effect of calcium ion concentration on GBS phage lysin activity.

FIG. 7 shows the effect of pH on GBS phage lysin activity.

### DETAILED DESCRIPTION

As used in this disclosure, the term “GBS phage lysin” refers to a polypeptide isolated from a GBS-specific phage that is capable of degrading bacterial cell walls as described herein, or a functional derivative thereof. GBS phage lysin may be encoded by SEQ ID NO: 1, or functional derivatives thereof, and may have the sequence of SEQ ID NO: 2, or functional derivatives thereof. The isolated and purified nucleic acid molecule which encodes GBS phage lysin is shown in SEQ ID NO. 1 and the isolated and purified nucleic acid sequence, along with flanking regions, is given in SEQ ID NO: 3 (GenBank accession number AY149214). The present disclosure is directed to GBS phage lysin nucleic acid having the sequence of SEQ ID NO: 1, as well as functional derivatives thereof. The predicted amino acid sequence of GBS phage lysin is shown in SEQ ID NO: 2. The present disclosure is directed to polypeptide having the sequence of SEQ ID NO: 2, as well as functional derivatives thereof. Also included in the scope of the present invention are nucleic acids which are at least 50% identical, 80% identical, 90% identical, or greater, to the nucleic acid sequence set forth in SEQ ID NO: 1, or functional derivatives thereof. Also included in the scope of the present invention are nucleic acids which encode amino acid sequences which are at least 50% identical, 80% identical, 90% identical, or greater to the amino acid sequence set forth in SEQ ID NO: 2, or functional derivatives thereof. The present disclosure also comprises partially purified or purified GBS phage lysin having the amino acid sequence of SEQ ID NO: 2, and functional derivatives thereof. Also included in the present disclosure are proteins which are about 50% identical, 80% identical, 90% identical, or greater to the amino acid sequences disclosed in SEQ ID NO: 2, and functional derivatives thereof. Furthermore the present disclosure comprises pharmaceutical preparations comprising an effective amount of any of the above described nucleic acids and/or proteins. Also included in the scope of the present disclosure are expression vectors comprising nucleic acids having the sequence of SEQ ID NO: 1, or functional derivatives thereof, encoding GBS phage lysin, and host cells comprising at least one of these expression vectors. Methods for creating and isolating anti-GBS phage lysin antibodies are contemplated in the present disclosure.

As used herein, the term “isolated” means that the molecule is removed from its native environment, e.g., a cell. For example, a naturally-occurring polynucleotide or polypeptide present in a living animal is not isolated, but the same polynucleotide or polypeptide, separated from some or all of the coexisting materials in the natural system, is isolated. Thus, an isolated molecule can be free of some or all cellular components, i.e., components of the cells in which the native material is occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified. Specifically exclude from this definition are molecules, such as nucleic acids, in mixtures of DNA molecules, transfected cells and cells clones as these occur in a DNA library such as a cDNA or genomic library.

The term “purified” does not require absolute purity; rather, it is intended as a relative definition. Purification of starting material or natural material to at least one order of magnitude, preferably two or three orders, and more preferably four or five orders of magnitude is expressly contemplated. In one embodiment, purified may refer to a polynucleotide or a polypeptide substantially free of contaminant. As used herein, the term “substantially free” is used operationally, in the context of analytical testing of the material. In one embodiment, purified material is substantially free of contaminants if it comprises about 50%, 60 to 90% weight/weight of a polypeptide or polynucleotide sample, respectively, or about 95%, and even over about 99% pure but, may be specified as any integer of percent between 50 and 100. Purity can be evaluated by chromatography, gel electrophoresis, immunoassay, composition analysis, biological assay, and other methods known in the art.

As used interchangeably herein, the terms “nucleic acid(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). Nucleic acid sequences referred to herein encompass the nucleic material itself and are thus not restricted to the sequence information (i.e. the succession of letters chosen among the four base letters) that biochemically characterizes a specific DNA or RNA molecule. Nucleic acid sequences also encompass those sequences containing “modified nucleotides” which comprise at least one modification such as (a) an alternative linking group, (b) an analogous form of purine, (c) an analogous form of pyrimidine, or (d) an analogous sugar (see, e.g., WO 95/04064). The nucleic acids sequences of the invention may be prepared by any known method, including synthetic, recombinant, ex vivo generation, or a combination thereof, as well as utilizing any purification methods known in the art.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini, and may be present in the same or varying degrees at several sites in a given polypeptide. Also, a given polypeptide may contain many types of modifications. Polypeptides may be branched, for example, as a result of ubiquitination, and they may be cyclic, with or without branching. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

The terms “complementary” or “complement thereof” are used herein to refer to the sequences of polynucleotides which is capable of forming Watson & Crick base pairing with another specified polynucleotide throughout the entirety of the complementary region. For the purpose of the present disclosure, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”.

As used in this disclosure, the term “percent homology” of two amino acid sequences or of two nucleic acid sequences is determined using the algorithm of Karlin and Altschul (Karlin and Altschul, 1990. Proc. Natl. Acad. Sci. USA 87:22264-2268) modified as in Karlin and Altschul (Karlin and Altschul, 1993. Proc. Natl. Acad. Sci. USA 90:5873-5877). Such an algorithm is incorporated into the NBLAST and XBLAST programs of Altschul et al. (Altschul, et al., 1990. J. Mol. Biol. 215:403-410). Blast nucleotide searches are performed with the NBLAST program, score=100, wordlength=12, to obtain nucleotide sequences homologous to a nucleic acid molecule of the invention. Blast protein searches are performed with the XBLAST program, score=50, wordlength=3 to obtain amino acid sequences homologous to a referenced polypeptide. To obtain gapped alignments for comparison purposes, Gapped BLAST is utilized as described in Altschul et al. (Altschul, et al. Nucleic Acids Res. 25:3389-3402). When utilizing BLAST and Gapped BLAST programs, the default parameters of the respective programs (XBLAST and NBLAST) are used. See http://www.ncbi.nlm.nih.gov.

The term “functional derivatives” includes “fragments,” “degenerate variants,” “mutants,” “variants,” and “chemical derivatives.” When referring to a polypeptide, the term “fragment” is meant to refer to any polypeptide subset of GBS phage lysin having the amino acid sequence shown in SEQ ID NO:2. In one embodiment, the fragment is at least 5 amino acids in length; however, the fragments may be of greater length in alternate embodiments. In one embodiment, the polypeptide fragment may contain the AXE domain (amino acids 6-107), the Acm domain (amino acids 145-344), or both. Such polypeptide fragments may be used as immunogens to generate antibodies reactive against the full length GBS phage lysin protein. Fragments of the disclosure may be assayed for GBS phage lysin activity using the methods described herein. When referring to a polynucleotide, the term “fragment” is meant to refer to any nucleic acid subset of a nucleic acid sequence encoding GBS phage lysin as shown in SEQ ID NO: 1. In one embodiment, the fragment is at least 15 nucleotides in length; however, the fragments may be of greater length in alternate embodiments. In one embodiment, the polynucleotide fragment may code the AXE domain, the Acm domain, or both. Such polynucleotide fragments can be used as PCR primers and hybridization probes. It is known that there is a substantial amount of redundancy in the nucleic acid codons which code for specific amino acids. For example, the nucleotide sequence of SEQ ID NO: 1, or a fragment thereof, could be altered to include alternative codons such that the polypeptide encoded by SEQ ID NO: 1, or a fragment thereof, is not functionally altered. In one embodiment, the alternative codons could encode for the same amino acid. In an alternate embodiment, the alternative codon could direct a conservative amino acid change. A conservative amino acid change, includes, but is not limited to, substitution of valine for leucine or asparagine for glutamine. Other conservative amino acid changes include the substitution on one amino acid for another amino acid contained in the same group (apolar side chain groups, polar side chain groups and charged side chain groups) as set forth in Biochemistry, Zubay, 2cd edition, McMillian Publishing Company, New York (Table 1.1). Therefore, this disclosure is directed to “degenerate variants” of the nucleic acid sequence of SEQ ID NO: 1, or a fragment thereof, and the amino acid sequence of SEQ ID NO: 2, or a fragment thereof. Referring to a polynucleotide, a degenerate variant includes those nucleic acid sequences which contain alternative codons which code for the eventual translation of the identical amino present in the base sequence or a conservatively changed amino acid. Referring to a polypeptide, a degenerate variant refers to those polypeptides having conservative amino acid substitutions as compared to the base sequence. It is known that DNA sequences coding for a polypeptide may be altered so as to code for a peptide having properties that are different than those of the naturally-occurring peptide (referred to as “mutants”). Mutant sequences may be isolated from nature or produced using methods known to those of skill in the art. Methods of producing mutant nucleic acid sequences include, but are not limited to site directed mutagenesis. Examples of altered properties include, but are not limited to changes in the affinity of an enzyme for a substrate, a receptor for a ligand or altered enzymatic properties and altered modification patterns. Included in the definition of a mutant are nucleotide sequences that encode a polypeptide wherein the cysteine at position 26 is replaced by serine, the cysteine at position 44 is replaced by serine, or both cysteine residues are replaced by serine. The term “variant” is meant to refer to a molecule substantially similar in structure or function to either SEQ ID NO: 1 or SEQ ID NO: 2, or to fragments thereof. Therefore, if the two molecules possess substantially similar activity, they are considered to be variants even if the structure of one of the molecules is not found in the other, or even if the two amino acid or nucleic acid sequences are not identical. Such variants may be identified using nucleic acid probes derived from “fragments” discussed above, or using antibodies discussed below. The term “chemical derivative” describes a nucleic acid or protein that contains additional chemical moieties which are not normally a part of the base molecule. Such moieties may alter the solubility, binding characteristics (to molecules such as growth factors) half-life, absorption, etc. of the base molecule. Alternatively the moieties may attenuate undesirable side effects of the base molecule or decrease the toxicity of the base molecule. Examples of such moieties are described in a variety of texts, such as Remington The Science and Practice of Pharmacy, 20^{th} edition.

Nucleic acid encoding for GBS phage lysin may be used to isolate and purify homologues of nucleic acids coding for homologues of GBS phage lysin from other organisms. To accomplish this, GBS phage lysin nucleic acid may be mixed with a sample containing nucleic acids encoding homologues of lysin under appropriate hybridization conditions. The hybridized nucleic acid complex may be isolated and the nucleic acid encoding the homologous target may be purified there from. Because the genetic code is degenerate, more than one codon may be used to encode a particular amino acid, and therefore, the same amino acid sequence can be encoded by any of a set of similar oligonucleotides. Only one member of the set will be identical to the GBS phage lysin nucleic acid sequence, and will be capable of hybridizing to GBS phage lysin nucleic acid, under appropriate conditions, even in the presence of oligonucleotides with mismatches. Under alternate conditions, the mismatched oligonucleotides may still hybridize to the GBS phage lysin nucleic acid to permit identification and isolation of GBS phage lysin homologues.

The disclosure also includes nucleic acids that hybridize under stringent conditions (as defined herein) to at least a portion of the nucleotide sequence represented by SEQ ID NO:1, or its complement. The hybridizing portion of the hybridizing nucleic acid is generally 15-50 nucleotides in length. Hybridizing nucleic acids as described herein can be used for many purposes, such as, but not limited to, a cloning probe, a primer for PCR and other reactions, and a diagnostic probe. Hybridization of the hybridizing nucleic acid is typically performed under stringent conditions. Nucleic acid duplex or hybrid stability is expressed as the melting temperature Tm, which is the temperature at which the hybridizing nucleic acid disassociates with the target nucleic acid. This melting temperature is many times used to define the required stringency conditions. If sequences are to be identified that are related to and/or substantially identical to the nucleic acid sequence represented by SEQ ID NO: 1, rather than identical, then it is useful to establish the lowest temperature at which only homologous hybridization occurs with a particular concentration of salt (such as SSC or SSPE). Assuming that 1% mismatch results in a 1° C. decrease in Tm, the temperature of the final wash in the hybridization reaction is reduced accordingly (for example, if a sequence having a 90% identity with the probe are sought, then the final wash temperature is decreased by 5° C. The change in Tm can be between 0.5° C. and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. The parameters of salt concentration and temperature can be varied to achieve the optimal level of identity between the probe and the target nucleic acid. Additional guidance regarding such conditions is readily available in the art.

GBS phage lysin, or functional derivatives thereof, may be recombinantly expressed either alone or in combination with a protein or nucleic acid that modulates the function of the GBS phage lysin, or its functional derivatives (a “modulating compound”), by molecular cloning into an expression vector containing a suitable promoter and other appropriate transcription regulatory elements, and transferred into prokaryotic or eukaryotic host cells to produce recombinant molecules. Techniques for such manipulations are within the ordinary skill of one in the art, and representative techniques can be found described in Sambrook, J., et al., Molecular Cloning Second Edition, 1990, Cold Spring Harbor Press. Expression vectors are defined herein as the nucleic acid sequences that are required for the transcription of genes and the translation of their mRNAs in an appropriate host. Such vectors can be used to express genes in a variety of hosts such as bacteria, blue green algae, plant cells, insect cells, fungal cells and animal cells. Specifically designed vectors allow the shuttling of DNA between hosts such as bacteria-yeast, or bacteria-animal cells, or bacteria-fungal cells, or bacteria-invertebrate cells. An appropriately constructed expression vector should contain, at the minimum: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. Expression vectors may include, but are not limited to, cloning vectors, modified cloning vectors, specifically designed plasmids or viruses.

A variety of expression vectors may be used, including, but not limited to, mammalian expression vectors, bacterial expression vectors and insect expression vectors. The expression vectors may be obtained from various commercial suppliers or produced according to specific needs. The choice of the appropriate expression vector is within the ordinary skill of one in the art. The expression vector may contain nucleic acid coding only for the GBS phage lysin, or a function derivative thereof, or may encode for the GBS phage lysin, or a functional derivative thereof, either alone or in combination with a modulating compound.

Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including, but not limited to, Drosophila and silkworm derived cell lines. A variety of cell lines derived from mammalian species which may be suitable for use as host cells are commercially available. The choice of host cells is within the ordinary skill of one in the art.

The expression vectors may be introduced into host cells via any one of a number of techniques including but not limited to transformation, transfection, lipofection, protoplast fusion, and electroporation. The expression vector-containing cells are clonally propagated and individually analyzed to determine whether they produce the compound of interest. Identification of cells expressing the GBS phage lysin protein, or a functional derivative thereof, can be accomplished by a variety of means, including but not limited to, immunological reactivity, or the presence of host cell-associated GBS phage lysin activity.

Expression of GBS phage lysin, or functional derivatives thereof, either alone or in combination with a modulating compound may also be performed using in vitro produced synthetic mRNA or isolated native mRNA. Synthetic mRNA or mRNA isolated from GBS phage lysin producing cells can be efficiently translated in various cell-free systems, including but not limited to wheat germ extracts and reticulocyte extracts, as well as efficiently translated in cell based systems, including but not limited to microinjection into frog oocytes.

### Bacterial Susceptibility

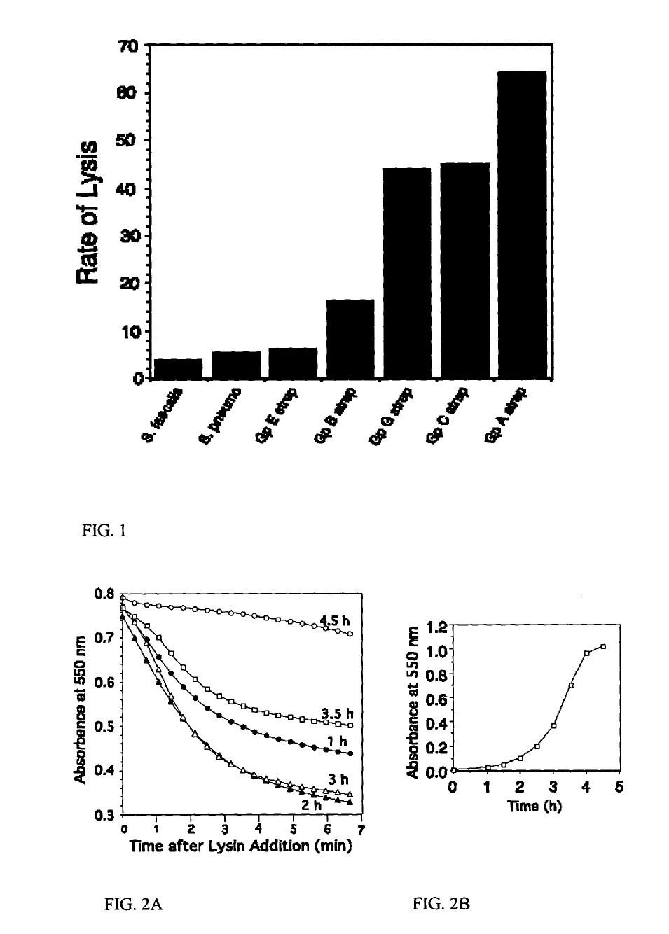

As determined by the in vitro bactericidal assay described blow, bacteria sensitive to lysis by GBS phage lysin include, but are not limited to, Streptococci from Groups B, C, G, A, and E (the hemolytic streptococci) (FIG. 1). The ability of the GBS phage lysin to lyse cells of GBS and other streptococcal species and gram positive bacteria is shown in FIG. 1 and Table 1. In all cases, the test bacteria were harvested by centrifugation after they had reached an absorbance of 0.4, and resuspended in lysin buffer A to an absorbance of 0.8 as described below. As shown in FIG. 1, group A streptococci were most sensitive to lysis by the GBS phage lysin followed by groups C and G streptococci. GBS strain 3331 (group B) was lysed only about one quarter as rapidly as group A streptococci. S. faecalis, group E streptococci, and an autolysis negative mutant of S. pneumoniae (LytA.) were lysed very slowly. GBS phage lysin was without effect on the bacteria listed in Table 1. As shown in Table 1, Lactobacillus are not sensitive to lysis by GBS phage lysin. This result is important since in one application described in this disclosure, Lactobaccillus strains are used as commensal bacteria for expression of GBS phage lysin as a bacteriocide. This result demonstrates that the protein itself has no deleterious effect on such “delivery” bacteria. This specificity regarding lysis of certain bacteria and not others by GBS phage lysin is linked to the composition of the interpeptide bridges that help form the peptidoglycan structure in these organisms (discussed in more detail below).

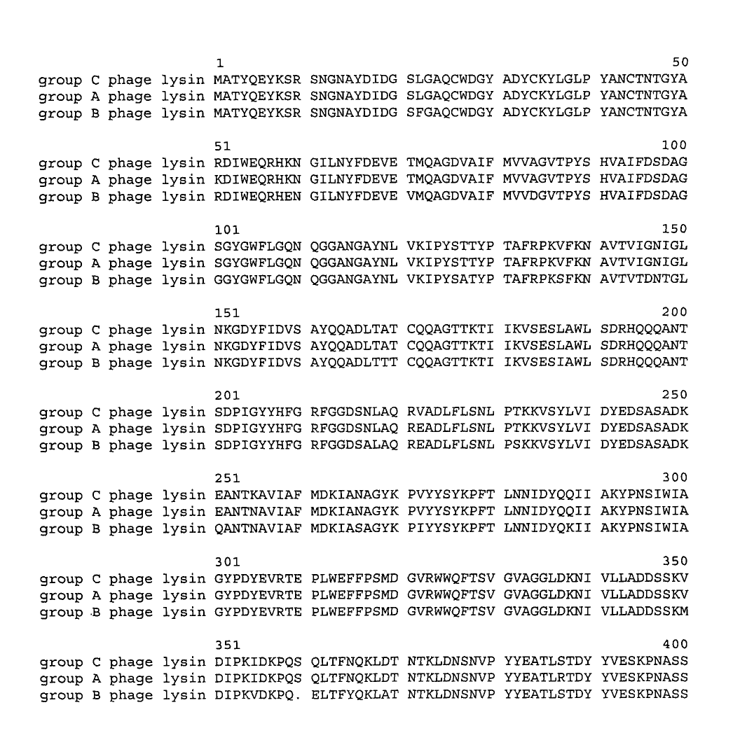

The susceptibility of bacterial cells to lysis is dependent on the growth stage at which the cells are harvested. Stationary phase cells appeared to be a poorer substrate than early log phase cells. This observation was confirmed by removing aliquots of a freshly inoculated culture of strain 3331 GBS cells at defined intervals during growth, washing the cells, and resuspending them to give an A₅₅₀ nm of approximately 0.8. The rate of lysis of the suspensions was then determined after addition of a fixed amount of the GBS phage lysin. As shown in FIG. 2A, GBS cells harvested in early log phase (2 to 3 h after inoculation) were lysed more rapidly than cells in late log phase (3.5 h) or stationary phase (4.5 h). Resistance to lysis did not increase further after overnight growth. FIG. 2B shows the growth curve of the GBS strain 3331 cells from which the aliquots were removed. The reason for this dependence is not clear, but it may be due to modifications of the cell wall that take place in stationary phase culture that make the bacteria more resistant to lysis. Modifications that may be important include increased cross-linking of the peptidoglycan layer and an increase in the amount of cell wall-associated proteins and polysaccharides. Heating GBS cells or cell wall preparations in boiling water made them poorer substrates for the lysin. Again, denatured cell wall-associated proteins may be impeding access of the lysin to the peptidoglycan. For the purposes of the disclosure, this difference in rate of lysis is not meant to suggest that GBS phage lysin will not be effective against cells in later stages of the growth cycle since the GBS phage lysin will be present for a significant period of time, much longer than the 6 minute time frame in this particular experiment. One can also argue that the remaining unkilled bacteria would increase their growth rate because of the lessened competition for nutrients, and therefore become more susceptible to GBS phage lysin.

GBS peptidoglycan consists of a glycan backbone of alternating β-1,4-linked Nacetylglucosamine and β-1,4-linked N-acetylmuramic acid residues linked to a stem peptide composed of L-Ala-D-iGln-L-Lys-D-Ala-D-Ala (Schleifer, et al. 1972, Bacteriol. Rev. 36: 407-477). The stem peptides are cross-linked by an (L-Ala)₂ or L-Ala-L-Ser interpeptide bridge between the ε-amino group of the L-Lys of one stem peptide cross-bridge and the carboxylate of a D-Ala of another stem peptide (Karakawa, et al., 1966; J. Exp. Med. 124: 155-171; Reinscheid, et al., 2002, Microbiology 148: 3245-3254). This linkage is characteristic of an A3α type of peptidoglycan in the classification system described by Schleifer and Kandler (Schleifer, et al., 1972). Phage lysins can potentially degrade peptidoglycan by cleaving any of several different linkages (Young, et al., 2000, Trends Microbiol. 8: 120-128). Some lysins are glycosidases (e.g. N-acetylmuramidases or N-acetylglucosaminidases). Other lysins possess specific endopeptidase activity. Another common type of lysin is an N-acetylmuramyl-L alanyl amidase that cleaves the linkage between the stem peptide and N-acetylmuramic acid of the glycan chains.

Analysis of conserved domains within the predicted open reading frame for the GBS phage lysin gene using the CDD search system at NCBI (Marchler-Bauer, et al., 2002, Nucleic Acids Res. 30: 281-283.; Marchler-Bauer, et al., 2003, Nucleic Acids Res 31: 383-387.) revealed two conserved domains. An AXE domain (pfam05257.1; amino acids 6 to 107) is present at the amino terminus and an Acm domain (COG3757.1; amino acids 145-344) is located in the central part of the protein. The Acm domain is associated with a lysozyme-type activity in other proteins. To determine if GBS phage lysin possessed glycosidase activity consistent with lysozyme activity, GBS cell walls were prepared and digested with GBS phage lysin for 4 h at 30° C., while monitoring the extent of digestion by turbidimetry (Table 2) as described below. An aliquot of the digest was then assayed for reducing groups using a modified Park-Johnson method (Spiro, R. G. 1996, Methods Enzymol. 8: 3-26). An increase in reducing activity would be expected if glycosidic bonds ere being cleaved by the GBS phage lysin. Digestion was clearly accompanied by an increase in reducing groups (Table 2), compared to a negligible level in the cell wall control (Table 2). This liberation of reducing groups is consistent with the GBS phage lysin cleaving glycosidic bonds in the repeating disaccharide backbone of the peptidoglycan. The glycosidase activity has been isolated to a 20 kDa fragment that is C-terminal to the AXE domain of the GBS phage lysin.

The second conserved domain in the GBS phage lysin is an AXE domain. The AXE domain is similar to or identical to the CHAP domain that has recently been described (Bateman and Rawlings, 2003, Trends Biochem Sci 28: 234-237; Rigden et al., 2003, Trends Biochem Sci 28: 230-234). The AXE domain is also present in a lysin of staphylocococcal phage φ11, where it was shown to be responsible for D-alanyl-glycine endopeptidase activity (Navarre, et al., 1999). To determine whether the GBS phage lysin also exhibits endopeptidase activity, the soluble digestion products from a GBS cell wall digest were analyzed by N-terminal amino acid sequencing as described below. Alanine was identified as the predominant residue through 3 cycles of sequencing. Alanine was also seen in the cell wall control through cycles 1-3, but at much lower levels. Sequencing the insoluble cell wall residues remaining after digestion again showed alanine as the only major N terminal residue, but the residue from the lysin digest contained significantly less N-terminal alanine than the cell wall control digest. One possible explanation for this result is that the glycosidase activity present in the GBS phage lysin yielded soluble muropeptide fragments possessing N-terminal alanine residues. In order to determine whether N-terminal alanine appearing in the soluble fraction was the direct result of endopeptidase activity, cell walls were acetylated to block pre-existing free amino groups and subsequently incubated with the lysin. The acetylated cell walls remained a satisfactory substrate for the lysin, as measured by a drop in turbidity during incubation (Table 2). N-terminal peptide sequencing of an ultrafiltrate of the digest again showed alanine as the predominant residue through cycles 1-3 (Table 2), and the acetylated cell wall control gave only negligible levels of alanine through 3 cycles of sequencing. These results show that the GBS phage lysin possesses endopeptidase activity in addition to its glycosidase activity.

The cysteine residues at position 26 and 44 of SEQ ID NO: 2 are critical for the endopeptidase activity of the GBS phage lysin. Mutation of either of these cysteine residues to serine abolishes endopeptidase activity of the GBS phage lysin. Glycosidase activity is not inhibited by these mutations. Mutations of the cysteine residues at position 34 and 171 do not inhibit endopeptidase activity or glycosidase activity of the GBS phage lysin.

The N-terminal AXE domain present in the GBS phage lysin is currently described as a type of anidase domain by GenBank (Bollinger et al., 1995, J. Biol Chem 270: 14031-14041). The AXE domain also is present in a lysin of staphylococcal phage φ11, where it was shown to be responsible for D alanyl-glycine endopeptidase activity (Navarre et al., 1999). A separate central domain (amidase-2) conferring N-acetylmuramyl-Lalanyl amidase activity was also present in this bifunctional phage lysin. The ability of the staphylocococcal phage φ11 lysin to release N-terminal alanine residues, in addition to the glycine residues of the pentapeptide bridge, was considered to be evidence of N-acetylmuramyl-Lalanyl amidase activity in the enzyme (Navarre et al., 1999). Using N-terminal amino acid sequencing, we also found that lysis was accompanied by an increase in N-terminal alanine residues. However, the GBS phage lysin lacks an N-acetylmuramyl-L-alanyl amidase domain. The N-terminal alanine residues released must be generated by another mechanism. The presence of the AXE domain in the GBS phage indicates the increase in N-terminal alanine residues is that they result from endopeptidase activity cleaving between D-Ala of stem peptides and L-Ala of the interpeptide bridges. Although the endopeptidase of phage φ11 lysin cleaves between the D-Ala of the stem peptide and a glycine residue of the (gly)₅ bridge in the staphylocococcal peptidoglycan, such pentaglycine bridges are not present in GBS peptidoglycan. Instead (L-Ala)₂ or L-Ala-L-Ser interpeptide bridges are present. An endopeptidase cleaving at the N-terminus of a D-Ala in GBS peptidoglycan would expose N-terminal L-Ala residues. A lysin possessing such a D-alanyl-L-alanine endopeptidase activity has not been described previously.

Several other β-hemolytic streptococci that are lysed by the GBS phage lysin, including those of groups A, C, E, and G streptococci, also possess (L-Ala)₂ interpeptide bridges (Schleifer, et al., 1972). Conversely, Streptococcus mutans and related organisms, which are not lysed by GBS phage lysin, have been reported to possess L-Thr-L-Ala cross bridges (Schleifer, et al., 1972). The C₁ phage lysin, previously shown to specifically kill streptococci of groups A, C, and E, but not streptococci of groups B and G (Nelson, et al., 2001, Proc. Natl. Acad. Sci. USA 98: 4107-4112), was recently sequenced (Nelson, et al., 2003, J.Bacteriol 185: 3325-3332). It showed no significant homology to the GBS phage lysin described in the present disclosure. Although the C₁ phage lysin possesses amidase activity (Fischetti, et al. J.Exp.Med. 133: 1105-1117; Nelson et al., 2003), it does not ossess any known conserved sequence domains. Our results suggest that the GBS phage lysin, like the staphylocococcal phage φ11 lysin, is a bifunctional enzyme possessing two different types of lytic activity. The recent demonstration by Loeffler and Fischetti of a synergistic effect of combining two different types of phage lysins suggests that the presence of two different peptidoglycan-cleaving activities in one enzyme may also be particularly efficient at lysing bacterial cell walls. Such a bifunctional phage lysin enzyme may have advantageous properties in eliminating bacterial organisms of interest.

### Diagnostic Methods

Currently, semi-purified lysin preparations are used to disrupt streptococcal cell walls to expose a range of bacterial antigens to detection, such as via antibodies. GBS phage lysin can be used in diagnostic applications to determine the presence of a particular bacteria, particularly where greater quantitation or reduction in cross-reacting impurities improves the testing results. GBS phage lysin, maintained in enzymatically active form, can be prepared in diagnostic kits, e.g., by storage in an appropriate container (glass or plastic vial, in solution or as a dried preparation), along with a detection system, e.g., a bacterial antigen-specific immunoassay.

Described herein is a diagnostic test for the identification of bacteria, including but not limited to, Streptococci from Groups B, C, G, A, and E (the hemolytic Streptococci), from infected tissues. One particular advantage of the diagnostic kit described herein is the ability to GBS phage lysin to disrupt the cell walls of all of the above hemolytic Streptococci. As a result, the diagnostic kit and method described herein will allow the detection and diagnosis of bacterial infection caused by any of the above hemolytic Streptococci. The diagnostic kits and method described herein will also allow the detection of other bacteria having cell wall susceptible to GBS phage lysin action, such as those bacteria possessing peptidoglycan having (L-Ala)₂ or L-Ala-L-Ser interpeptide bridges, may also be identified. Since antigen from the bacteria is efficiently released from the cells in the sample, the identification test may be performed in a single step. In addition, the test provides the user with an answer in a short time without the need for complicated equipment or experience. This permits the test to be performed in both the doctor's office as well as the home. Thus, a health care professional is able to determine the course of treatment rapidly without the need to delay 24 to 48 hours for the results of conventional assays which require the growth of bacteria in the laboratory.

In one embodiment, the specimen is collected onto an means for collecting, such as but not limited to, an applicator stick fitted at one end with a fiber swab. The infected area is swabbed to transfer the organisms from the infected tissue to the swab. The swab is then transferred to a solution containing the purified GBS phage lysin enzyme, or a functional derivative thereof, in a buffered solution. In one embodiment, the functional derivative may be a polypeptide fragment having one or more of the activities associated with the GBS phage lysin (lysozyme or endopeptidase). The assay can be performed at room temperature. The enzyme digests the cell wall of a bacterium susceptible to GBS phage lysin that is present in the swab and releases the antigens, such as, but not limited to, group-specific carbohydrates, into solution. As antigen is released from a swab containing the susceptible bacteria, the means for detection, such as, but not limited to, antibodies and nucleic acid probes, will react with the antigen.

### Production of Antibodies

According to the present disclosure, GBS phage lysin, or functional derivatives thereof, produced recombinantly or by chemical synthesis may be used as an immunogen to generate antibodies that recognize the GBS phage lysin, or functional derivatives thereof. Such antibodies include but are not limited to polyclonal, monoclonal, chimeric, single chain, Fab fragments, and an Fab expression library. The anti-GBS phage lysin antibodies may be cross reactive, e.g., they may recognize GBS phage lysin or fragments thereof from different species.

Various procedures known in the art may be used for the production of polyclonal antibodies to polypeptides. For the production of antibody, various host animals can be immunized by injection with GBS phage lysin, or functional derivative thereof, including but not limited to rabbits, mice, rats, sheep, goats, etc. In one embodiment, the GBS phage lysin or functional derivatives thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies, any technique that provides for the production of antibody molecules by continuous cell lines in culture may be used. These include but are not limited to the hybridoma technique originally developed by Kohler and Milstein (Kohler and Milstein, 1975. Nature 256: 495-497), as well as the trioma technique, the human B-cell hybridoma technique (Kozobr, et al., 1883. Immunology Today 4: 72-78; Cote, et al., 1983. Proc. Natl. Acad. Sci. U.S.A. 80:2026-2030), and the EBV-hybridoma technique to produce human monoclonal antibodies (Cote, et al., in Monoclonal Antibodies and Cancer Therapy, Alan R. Liss, Inc. pp. 77-96, 1985). In an additional embodiment of the disclosure, monoclonal antibodies can be produced in germ-free animals (International Patent Publication No. WO 89/12690, published Dec. 28, 1989). In fact, according to the invention, techniques developed for the production of “chimeric antibodies” (Morrison, et al., 1984. J.Bacteriol. 159:870-875; Neuberger, et al., 1984. Nature 312:604-608; Takeda, et al., 1985. Nature 314:452-454) by splicing the genes from a mouse antibody molecule together with genes from a human antibody molecule of appropriate biological activity can be used; such antibodies are within the scope of this disclosure. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the present disclosure, techniques described for the production of single chain antibodies (U.S. Pat. Nos. 5,476,786, 5,132,405 and 4,946,778) can be adapted to produce GBS phage lysin-specific single chain antibodies. Such single chain antibodies may also be directed to functional derivatives of the GBS phage lysin. Indeed, the nucleic acids encoding these single chain antibodies can be delivered for expression in vivo. An additional embodiment of the invention utilizes the techniques described for the construction of F_{ab} expression libraries (Huse, et al., 1989. Science 246:1275-1281) to allow rapid and easy identification of monoclonal Fab fragments with the desired specificity for GBS phage lysin polypeptides, or functional derivatives thereof.

Antibody fragments which contain the idiotype of the antibody molecule can be generated by known techniques. For example, such fragments include but are not limited to: the F_{ab}² fragment which can be produced by pepsin digestion of the antibody molecule; the F_{ab} fragments which can be generated by reducing the disulfide bridges of the F_{ab}² fragment, and the F_{ab} fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a GBS phage lysin, a functional derivative thereof, or other lysin polypeptide, one may assay antibodies produced to determine which bind to a polypeptide fragment used to generate the antibody. The foregoing antibodies can be used in methods known in the art relating to the localization and activity, (e.g., Western blotting), imaging in situ, measuring levels in appropriate physiological samples using any of the detection techniques mentioned above or known in the art.

### Therapy and Administration

The antibiotic activity of GBS phage lysin, or functional derivatives thereof, advantageously permits treatment of bacterial infection in any animal, particularly mammals, and more particularly humans. The bacteria susceptible to GBS phage lysin, or functional derivatives thereof, treatment include any bacteria having a cell wall composition subject to the enzymatic action of GBS phage lysin, including, but not limited to, Streptococci from Groups B, C, G, A, and E. Bacteria containing peptidoglycan comprising (L-Ala)₂ or L-Ala-L-Ser interpeptide cross bridges have been shown to be susceptible to GBS phage lysin. Animals that can be treated include, but are not limited to, domesticated animals and pets (dogs, cats, rodents, ferrets, etc.), laboratory animals (rats, mice, rabbits, hamsters, guinea pigs, etc.), farm animals, and wild animals in natural and captive settings.

GBS phage lysin or functional derivatives thereof may be used therapeutically to inhibit the growth of bacteria comprising peptidoglycan which are susceptible to the activity of GBS phage lysin as stated above. In addition, GBS phage lysin or functional derivatives thereof may also be applied prophylactically to prevent a subject from becoming sick after an exposure to a potentially infectious bacterium, or to prevent reinfection of a bacteria after initial therapeutic treatment. Other prophylactic uses include use as a sterilization agent, or as applied to catheters, stents, artificial joins, pins, and other implanted devices, to prevent development of infections. In addition, GBS phage lysin or functional derivatives thereof may be implanted on the medical devices by including recombinant cells that produce the protein in the device, e.g., for coronary or peripheral arterial shunts.

In addition, GBS phage lysin or functional derivatives thereof may be applied in a variety of methods, including direct application, indirect application, via a carrier, or other means. The forms of administration include, but are not limited to, injectables, creams, ointments, powders, inhalable aerosols, candies, chewing gum, spray, mouthwashes/gargles and via incorporation in bandages and similar means. For example, GBS phage lysin or functional derivatives thereof may be incorporated into a bandage, or similar carrier, for application to a subject, such as over an open wound, abrasion or burn. Furthermore, the GBS phage lysin or functional derivatives thereof may be formulated such that it is released slowly over time from the bandage or similar carrier.

Furthermore, it is contemplated that modulating agents that increase the effectiveness of GBS phage lysin or functional derivatives thereof may be used in combination. For example, the holin or fragments thereof, may be used as a modulating agent to provide a therapeutic or prophylactic advantage. In this example, holin, may penetrate the bacterial cell membrane and increase the ability of GBS phage lysin to inhibit bacterial cells. In one embodiment, GBS phage lysin or functional derivatives thereof, either with or without modulating agents, may be used for the treatment and prevention of bacterial infections. The terms “preventing” as used herein refer to administering a compound prior to the onset of clinical symptoms of a disease or condition so as to prevent a physical manifestation of the disease or condition. The term “prophylaxis” is distinct from “treatment” and encompasses “preventing”. Prevention or prophylaxis need not be absolute to be useful. The term “treating” as used herein refers to administering a compound after the onset of clinical symptoms. The term “in need of treatment” as used herein refers to a judgment made by a caregiver that an individual or animal requires or will benefit from treatment. This judgment is made based on a variety of factors that are in the realm of a caregiver's expertise, but that include the knowledge that the individual or animal is ill, or will be ill, as the result of a condition that is treatable by a compound of the disclosure.

In one embodiment, the treatment and or prevention relates to the treatment and prevention of vaginal colonization, especially bacterial infections caused by GBS and related bacteria. Further discussion of this embodiment is given in Prophetic Example 1 below. In an alternate embodiment, the treatment and prevention relates to the treatment and prevention of infections caused by the hemolytic Streptococci. For example, Group A Streptococci may cause streptococcal pharyngitis (strep throat), impetigo, cellulites, erysipelas, puerperal fever (or sepsis), streptococcal toxic shock syndrome, necrotizing fasciitis. In addition, the toxins produced by some Group A Streptococci can also contribute to acute rheumatic fever and acute glomerulonephritis. In other embodiments, GBS phage lysin or functional derivatives thereof, either with or without modulating agents, may be used to treat other bacterial infection where the bacteria contain cell wall susceptible to GBS phage lysin enzymatic activity (i.e. those cell walls containing peptidoglycan containing interpeptide cross bridges of (L-Ala)₂ or L-Ala-L-Ser.

An effective amount of GBS phage lysin or functional derivative thereof may be recombinantly expressed and purified and applied directly to treat or prevent bacterial infections, or may be applied via an acceptable carrier. In the embodiment where vaginal colonization is treated or prevented, the carrier may be a pad, tampon or douche. In addition, nucleic acids encoding GBS phage lysin, or a functional derivative thereof, may be placed in expression vectors in recombinant host cells for expression of an effective amount of GBS phage lysin or functional derivatives thereof. Modulating compounds may be included in such recombinant host cells and be co-expressed with GBS phage lysin, or functional derivatives thereof.

Pharmaceutically useful compositions comprising GBS phage lysin, or functional derivatives thereof, and/or nucleic acid sequences coding for the same (“therapeutic agent”), may be formulated according to known methods such as by the admixture of a pharmaceutically acceptable carrier. Examples of such carriers and methods of formulation may be found in Remington The Science and Practice of Pharmacy, 20^{th} edition. To form a pharmaceutically acceptable composition suitable for effective administration, such compositions will contain an effective amount of the therapeutic agent. An “effective amount”, in reference to the treatment of a disease or condition, refers to an amount of a compound that is capable of having any detectable, positive effect on any symptom, aspect, or characteristics of the disease or condition. The effective amount may vary according to a variety of factors such as the individual's condition, weight, sex and age. Other factors include the mode of administration. The pharmaceutical compositions may be provided to the individual by a variety of routes such as subcutaneous, topical, oral and intramuscular. The therapeutic agents identified according to the methods disclosed herein may be used alone at appropriate dosages defined by routine testing in order to obtain optimal effects while minimizing any potential toxicity. In addition, co-administration or sequential administration of other agents may be desirable. The therapeutic agents discussed herein may be used with or without chemical derivatives.

All references to articles, books, patents, websites and other publications in this disclosure are considered incorporated by reference.

### Methods

### Bacterial Strains and Culture Conditions

GBS strains were stored at −70° C. in sheep blood and routinely plated on T-soy agar containing 5% sheep blood or cultured in Todd-Hewitt Broth (Difco) at 37° C. without shaking. Bacteriophages were detected and propagated on the solid media consisting of 30 g of Todd-Hewitt Broth base, 2 g of yeast extract, 12 mg CaCl₂, and 10 mg L-tryptophan per liter (Stringer, 1980). Groups A, C, E, and G streptococci, S. faecalis, S. mutans serotypes c (strain AHT), e (strain LM-7), f (strain DMZ 175), S. rattus (strains FA-1 and BHT) S. cricetus (strain AHT), S. sobrinus (strain SL-1), S. mitis, Staphylococcus aureus, were also grown in Todd-Hewitt Broth. The autolysis-deficient mutant of S. pneumoniae, L82016 (LytA.) was grown in Todd-Hewitt Broth containing 5 g yeast extract per liter. Lactobacillus casei, L. acidophilus, L. brevis, and L. delbrueki, kindly provided by Dr. Jane Schwebke, were grown in MRS Broth (Difco). Escherichia coli strains INVαF′ and BL21/DE3 were routinely grown with shaking in Luria-Bertani Broth (LB) at 37° C. or at 30° C. in Terrific Broth (TB) (4.8 g Bacto-tryptone, 9.6 g Bacto yeast extract, 2.35 g glycerol, adjusted after autoclaving to pH 7 with KH₂PO₄) for protein expression. Recombinant clones were grown in media supplemented with 50 ∝g/ml ampicillin.

### Cloning, Expression and Purification of GBS Phage Lysin

GBS strains often contain prophages that can be induced by various treatments. Serotype III GBS strain 3330 was exposed to mitomycin C (1 μg/ml) and bacteriophages were recovered. After centrifugation, culture supernates were filtered through a 0.2 μm filter and the filtrates were cross-spotted on lawns of the different GBS strains. Clear plaques in the lawns after overnight incubation indicated the presence of phage. One phage was selected, designated B30, for analysis. It was obtained from a mitomycin-treated culture of GBS serotype III strain 3330 and propagated in GBS serotype III strain 3331. It gave small clear plaques approximately 0.5 mm in diameter. Phage lysates were prepared by adding phage, at a multiplicity of about 10:1, to early log phase cultures of strain 3331 (A_{600 nm}=0.3). The cells lysed after about 1 hr at 37° C. Phage titers of approximately 10¹¹ pfu/ml were routinely obtained.

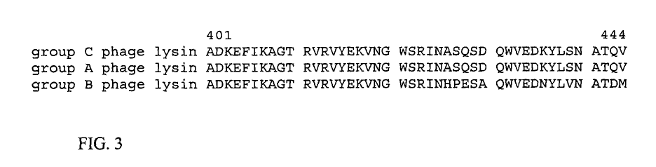

Phage particles were purified by precipitation with PEG 8000 followed by CsCl ultracentrifugation, and the DNA isolated using standard procedures (53). Plasmid libraries of phage DNA containing cloned segments of the entire phage genome were made in pUC 19 after cutting the phage DNA with Hind III and Bam H1. The inserts in the HindIII library were sequenced and a portion of a putative GBS phage lysin gene was detected in plasmid 35. An alignment of the predicted amino acid sequence of the cloned gene segment with protein sequences from a group C streptococcal phage lysin (Gp C Lysin), a putative group A streptococcal phage lysin (Gp A lysin), and the Cp-1 lysin of a pneumococcal phage (Spn Lysin) is shown in FIG. 3. Furthermore, despite the high sequence similarity in this region of the lysin gene, it is significant that both the pneumococcal and group C streptococcal phage lysins are highly specific and neither one lyses GBS (31, 36). It seems very likely that the GBS phage lysin also will be highly specific.

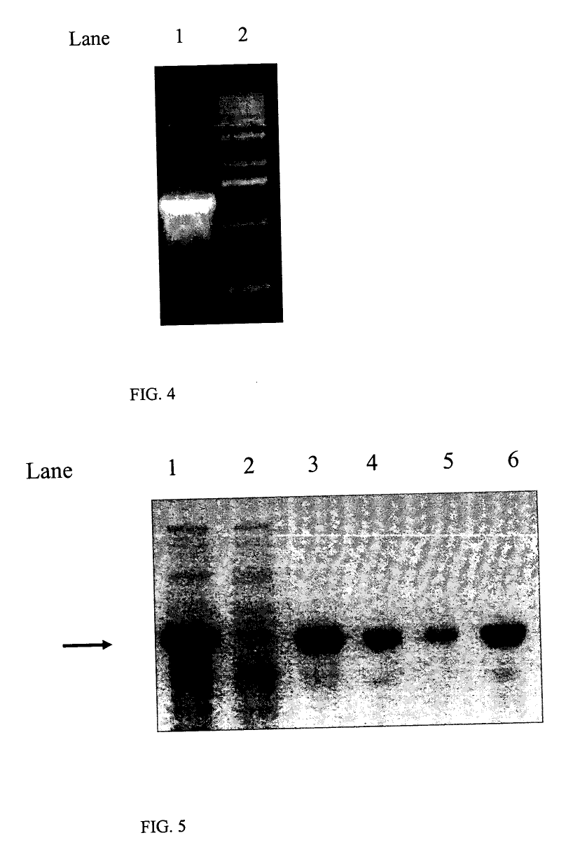

From the sequence of the gene segment above, polymerase chain reaction (PCR) was used to isolate the gene from phage DNA. The oligonucleotides used for PCR were 5′GCACTACATATGGCAACTTATCAAGAATATAAAAG3′ (forward) (SEQ ID NO: 4) and 5′GTGGTGCTCGAGCATATCTGTTGCGTTAACTAAGTAGC3′ (reverse) (SEQ ID NO: 5). Purified phage DNA was used as template in a PCR reaction and amplified with high fidelity Taq polymerase, according to manufacturer's instructions (PCR Supermix High Fidelity Taq polymerase, Gibco-BRL), for 35 cycles of 95° C. 45 seconds, 55° C. for 60 seconds, and 68° C. for 3 minutes. The product band was purified by gel electrophoresis on 0.8% low melt agarose (Sigma-Aldrich) and cloned into the pGEM T-vector (Promega, Madison, Wis.). The product band was 1300 bp as determined by gel electrophoresis (shown in FIG. 4). After cloning the purified plasmid DNA was cleaved with restriction endonucleases NdeI and XhoI and cloned into plasmid pET21a (Novagen) for expression. A few additional amino acids were added to the carboxyl-terminal end of the protein by sequences included in the pET21a plasmid, including a 6× histidine tag to aid with affinity purification. The resulting plasmid was checked by DNA sequencing to assure that the cloned sequence was correct.

The complete nucleic acid sequence of the GBS phage lysin gene and the flanking sequences is shown in SEQ ID NO: 3 (GenBank accession no. AY149214). The sequence of the gene and flanking regions revealed both an upstream (nucleotides 66-99) and a downstream (nucleotides 1512-1539) stem-loop structure that could serve as transcription terminators. A putative ribosome binding site is present starting at 13 nucleotides prior to the predicted ATG start codon for the GBS phage lysin nucleic acid. There is also an in-frame TAA stop codon immediately prior to the ATG start, suggesting that the predicted lysin gene is not part of some multi-domain protein. The calculated molecular mass of the GBS phage lysin is 49,677 Da.

The expression plasmid pET21a was introduced into E. coli BL21(DE3) cells for expression by chemical transformation. Cells containing the plasmid were selected by inclusion of ampicillin (50 μg/ml) in growth medium. Cells were inoculated into Terrific Broth (TB) medium (4.8 g Bacto-tryptone, 9.6 g Bacto yeast extract, 2.35 g (1.6 ml) glycerol, adjusted after autoclaving to pH 7 with KH₂PO₄) containing ampicillin (50 μg/ml). At an OD₅₅₀ of 0.5, IPTG was added to a final concentration of 1 mM, and the cells were further grown for 3-4.5 hrs. Cells were collected by centrifugation and washed 3 times with lysis buffer (50 mM Na H₂PO₄ (pH.8.0), 300 mM NaCl, 10 mM imidazole). The cells were lysed by sonication 10 times for 30 seconds each time in ice (Fisher sonic dismemberator, model 300 at 60% maximum power); sufficient time was allowed between each sonication to allow the sample to cool to prevent heating the sample. The cell debris was removed by centrifugation and the resulting supernatant passed over a 10 ml Ni-NTA nickel affinity column (Qiagen), according to the manufacturer's instructions. The column was washed with 50 ml 50 mM Na H₂PO₄ (pH.8), 300 mM NaCl and 20 mM imidazole. The affinity purified protein was eluted from the column with 250 mM imidazole (in 50 mM Na H₂PO₄ (pH.8), 300 mM NaCl). The eluate was concentrated to 2 ml on a Centri-prep concentrator and buffer exchanged 3× with 50 mM ammonium acetate, 1 mM DTT, pH 6.2, to remove phosphate ions, and then once with lysin buffer A (50 mM ammonium acetate, 10 mM CaCl₂, 1 mM DTT, pH 6.2). By SDS-polyacrylamide gel electrophoresis, a single prominent band of apparent molecular weight of 51,000 was observed (shown in FIG. 5). The protein of 51 kDa rapidly reduced the turbidity of a suspension of GBS strain 3331 cells. GBS phage lysin activity in column fractions was measured as described below.

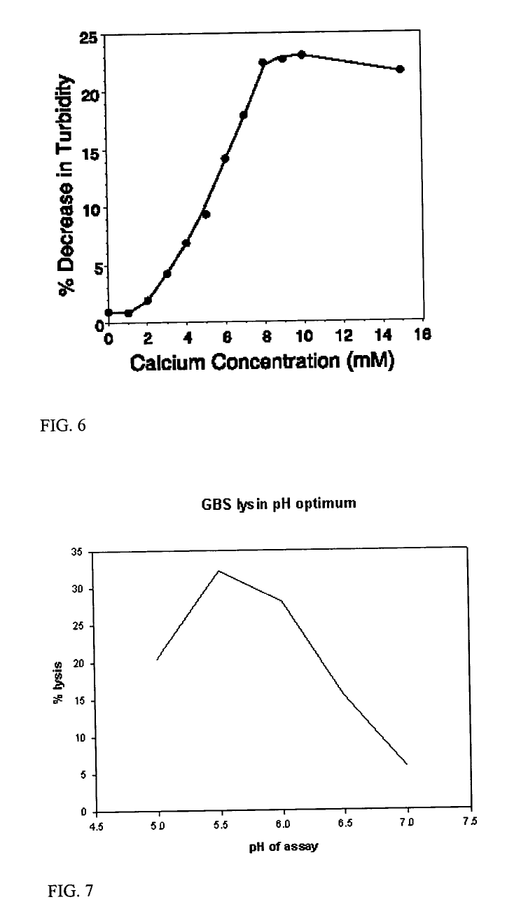

The effect of calcium ion concentration on GBS phage lysin activity was measured by as described below using a suspension of strain 3331 GBS cells. The assay was carried out at 30° C. in 50 mM ammonium acetate, 1 mM DTT, pH 6.2, containing different concentrations of CaCl₂. The optimum calcium ion concentration was determined to be 10 mM (FIG. 6). The pH optimum of the GBS phage lysin was determined in the presence of 10 mM CaCl₂, 1 mM DTT. The pH optimum of the purified GBS phage lysin is between 5.5 and 6.0 (FIG. 7).

### In Vitro Bacteriocidal Activity of GBS Phage Lysin

GBS phage lysin activity was determined by a turbidity reduction assay similar to that described by Nelson et al. (Nelson, et al., 2001). GBS phage lysin activity was measured by following the reduction in turbidity of a suspension of GBS cells after GBS phage lysin addition. A unit of activity is defined as the amount of enzyme that reduces the absorbance of 1 ml of a suspension of strain 3331 GBS cells (adjusted to an initial A₅₅₀ of 0.8) by 0.001/min at 30° C. Assays are carried out in lysin buffer A.

The susceptibility of other bacterial species to lysis by GBS phage lysin was also assessed by monitoring reduction in turbidity as discussed above. In all cases cells were grown to an A₅₅₀ nm of 0.4, harvested by centrifugation, washed 3× in lysin buffer A and resuspended in lysin buffer A to give an A₅₅₀ nm of 0.8. GBS phage lysin (10 μl, 25 u) was added to 0.4 ml of the suspension at 30° C. and the rate of decrease in turbidity during the first 2 minutes was measured.

### Preparation of Cell Walls

A crude cell wall preparation made from GBS strain 3331 cells was also a suitable substrate for the phage lysin. It was made by passing a suspension of GBS cells, grown to an A₅₅₀ nm of 0.4, through a French Press operated at approximately 15,000 p.s.i. Unbroken cells were removed by low speed centrifugation (5,000×g) for 5 min, and the cell walls were recovered from the supernate by centrifugation at 20,000×g for 45 min. They were washed three times in lysin buffer A. For studies of the glycosidase and endopeptidase activities of the GBS phage lysin, the cell walls were washed an additional three times with water. The final stock cell wall preparation, adjusted to an absorbance of 1.0 at 550 nm, was stored at 4° C. Cell walls were acetylated by a modified standard procedure (Riordan, et al., 1967. Methods Enzymol. 11: 565-570). Stock GBS cell walls in water (2.0 ml) were diluted with an equal volume of saturated aqueous sodium acetate at 4° C. and acetylated by adding 5×40 μl aliquots of acetic anhydride over a period of 1 h with constant stirring. The acetylated cell walls were washed 3 times with water, then diluted with water to give a suspension with a turbidity reading of 1.0 at 550 nm and stored at 4° C. A test for free amino groups using 2,4,6-trinitrobenzenesulfonic acid (Mokrasch, L. C., 1967, Analyt. Biochem. 18: 64-71) gave a negative reaction for acetylated cell walls (i.e., the cell walls remained white), whereas the nonacetylated cell walls were strongly positive (i.e., they became yellow-brown in color).

### Measurement of Endopeptidase Activity

The soluble products released from GBS cell walls by digestion with the phage lysin were analyzed by N-terminal amino acid sequencing. Incubation mixtures (see footnotes, Table 2) were clarified by centrifugation, the supernatants passed through Ultrafree-MC 5000 MW cutoff filters, and 20 μl aliquots applied to protein support disks for N-terminal sequencing. Insoluble cell wall residues from the same incubation mixtures were recovered by centrifugation, washed once with lysin buffer B (see footnotes, Table 2), twice with water, and an equivalent aliquot applied to a protein support disk. Cell wall controls, not digested with the lysin, were treated similarly. All samples were subjected to 3-4 cycles of Edman degradation and the released PTH amino acid(s) were identified following separation by RP-HPLC. Analyses were performed using a Beckman peptide microsequencer (Model PI 2090E).

### Measurement of Glycosidase Activity of GBS Phage Lysin

Lysin digests of cell walls were assayed for cleavage of the glycan using a modified Park-Johnson method (Spiro, 1966). Briefly, digests were clarified by centrifugation, when necessary, and aliquots (0.2 ml) were heated with 0.05% (w/v) aqueous potassium ferricyanide (0.2 ml) and 0.53% (w/v) sodium carbonate/0.065% (w/v) potassium cyanide in water (0.2 ml) at 100° C. for 15 min. Under these alkaline conditions, reducing groups liberated by the cleavage of glycosidic bonds in the peptidoglycan glycan chain will stoichiometrically reduce ferricyanide to ferrocyanide. Upon cooling, 1.0 ml of a ferric ion reagent (0.15% (w/v) ferric ammonium sulfate/0.1% (w/v) SDS in 0.025 M sulfuric acid) is added, which forms a stable prussian blue color. After 15 min at room temperature, the A₆₉₀ nm was measured. Data was converted to glucose equivalents using a standard curve.

### PROPHETIC EXAMPLE 1

### Use of GBS Phage Lysin to Treat and/or Prevent GBS Vaginal Colonization

In a large prospective study at The University of Alabama at Birmingham in 1987, it was found that the attack rate for neonatal disease was about 4 per 1000 live births (early- and late-onset infections), while maternal sepsis occurred in 1-2 per 1000 deliveries (Dillon, et al. 1987. J.Pediat. 110:31-36). Although the attack rate has been significantly reduced by the now prevalent use of intrapartum antibiotics, GBS remains the most important bacterial pathogen in perinatal infections (Centers for Disease Control and Prevention. 1996). GBS is also one of the most important causes of postpartum infections, causing amnionitis, wound infections, urinary tract infections, and bacteremia (Gibbs, et al. 1981. Am. J. Obstet. Gynecol. 140:405-411). GBS has also been reported to cause premature rupture of membranes and preterm delivery (Centers for Disease Control and Prevention., 1996; Regan, et al. 1981. Am. J. Obstet. Gynecol. 141:184-186).

Early-onset infections, which account for 81% of neonatal GBS disease (Zangwill, et al. 1992), are overwhelmingly the result of vertical transmission. The presence of GBS in the birth canal of the mother is the primary determinant of both asymptomatic colonization and life-threatening infections of the neonate (Yow, et al., 1980, Am. J. Obstet. Gynecol. 137:34-38). Unfortunately, vaginal carriage of GBS is very common. In one prospective study 21% of pregnant women were found to be vaginally colonized (Regan, et al., 1996, Am. J. Obstet. Gynecol. 174:1354-1360). This study also found that neonatal GBS sepsis occurred in 16 out of 1000 live births to women vaginally colonized with GBS at delivery (Regan, et al. 1996). Transmission rates from 42 to 72% have been reported for infants born to vaginally colonized mothers (Baker, et al. 1973, J.. Pediat. 83:919-925; Yow, et al., 1980). Infants were most likely to be colonized when born to mothers heavily vaginally colonized with GBS (Anthony, et al. 1978 J. Infect. Dis. 137:524-530; Regan, et al., 1996). The observation that the majority of infants with early-onset infections were bacteremic at birth and presumably infected in utero suggested an ascending route of infection from the vagina (Boyer, et al. 1983. S J. Infect. Dis. 148:795-801; Pyati, et al. 1983. N.Engl.J.Med.:1383-9). Therefore, any procedure that eliminates or reduces GBS vaginal colonization would be expected to greatly decrease the incidence of early-onset GBS infections.

Approaches involving antepartum antibiotics have not worked and treated women were often rapidly recolonized (Hall, et al. 1976. Am.J. Obstet. Gynecol. 124:630-634; Sweet, et al. 2002. P. 31-46, Infectious Diseases of the Female Genital Tract., Fourth ed. Lippincott William & Williams, Philadelphia). This may be because a reservoir of GBS in the GI tract is difficult to eliminate (Sweet, et al. 2002). Women also may be re-exposed by their sexual partners. Although implementation of currently recommended strategies for intrapartum antibiotic use (Centers for Disease Control and Prevention. 1996) has significantly decreased the incidence of early onset infections, the approach appears to have its limitations. For example, it was estimated that the use of intrapartum antibiotics based on risk factor assessment would prevent only 41% of early-onset infections (Rosenstein, et al. 1997. Group. Obstet. Cynecol. 90:901-906).

Another promising approach to preventing early-onset infections is maternal immunization with GBS polysaccharide or protein antigens. Transplacentally passed maternal antibodies would then presumably protect the neonate from GBS disease. However, this approach may not adequately protect the group of infants at highest risk, those born prematurely, since maternal antibody is passed late in pregnancy (Noya, et al. 1992. Infect Dis. Clin. North Am. 6:41-55). Conceivably, an immunization procedure that induced secretory antibody might limit GBS vaginal colonization, but this has not yet been demonstrated and there is no evidence that mucosal immunity develops naturally, despite long-term GBS colonization. Therefore, new approaches to preventing GBS infections are needed.

A completely different approach to treating and/or preventing GBS vaginal colonization is described in the current disclosure. In one embodiment, the treatment and/or prevention involves the use of a genetically engineered commensal containing GBS phage lysin nucleic acid. In one embodiment, the nucleic acid has the sequence of SEQ ID NO: 1. In an alternate embodiment, the nucleic acid is a functional derivative of the sequence of SEQ ID NO: 1. The commensal organism are engineered to express an effective amount of protein product. In an alternate embodiment, purified or semi-purified GBS phage lysin may be used directly to treat and/or prevent vaginal colonization of GBS.

The predominant commensal organism in the normal vaginal flora of women of reproductive age is the lactobacillus (Sobel, 1999. Curr. Infect. Dis. Rep. 1:379-383). It is now well established that these gram-positive bacilli play an important role in controlling the vaginal microenvironment and in preventing the overgrowth of potentially pathogenic organisms (Hawes, et al. 1996. J. Infect. Dis 174:1058-63; Klebanoff, et al. 1991. J. Infect. Dis 164:94-100). They do this by secreting a number of substances, including lactic acid, hydrogen peroxide, and a variety of bacteriocins (Klebanoff, et al. 1991). The lactic acid is responsible for maintaining a low vaginal pH, which inhibits the growth of many potential pathogens. The hydrogen peroxide is particularly important. In a study of 275 women in the second trimester of pregnancy it was found that the absence of H₂O₂-positive lactobacilli in the vagina was associated with a much higher incidence of bacterial vaginosus and colonization with Gardnerella vaginalis, Bacteroides, Peptostreptococcus, Mycoplasma hominis, Ureaplasma urealyticum, and Chlamydia trachomati (Hillier, et al. 1992. Obstet. Gynecol. 79:369-73). Interestingly, these authors found that colonization with group B streptococci was not related to the presence of H₂O₂-positive lactobacilli. GBS apparently are resistant to the antimicrobial factors secreted by the lactobacilli.

It is anticipated that the GBS phage lysin or functional derivatives thereof expressed from commensal organisms will specifically attack the cell walls of GBS and other Streptococci without significantly affecting the normal vaginal flora. As discussed above, GBS phage lysin did not lyse several Lactobacillus strains. Two recent reports demonstrated the ability of purified preparations of phage lysins to selectively destroy their bacterial targets. In one case nasopharyngeal colonization of mice by group A streptococci was eliminated by treatment with a purified lysin isolated from a group C streptococcus (Nelson, et al. 2001). The enzyme specifically killed groups A, C, and E streptococci but not 14 other normal commensal streptococci. It also did not affect GBS or streptococci belonging to groups D, F, G, L, and N. In the second report, nasopharyngeal colonization of mice by pneumococci was eliminated by treatment with a recombinant pneumococcal phage lysin (Loeffler, et al. 2001. Science 294:2170-2). This enzyme was highly specific for S. pneumonia. There are also examples of microorganisms secreting a peptidoglycan-degrading enzyme to prevent the growth of other bacteria. For example, lysostaphin, a bacteriocin produced by Staphylococcus simulans, specifically cleaves the pentaglycine cross-bridges in the peptidoglycan of other susceptible staphylococci (Baba, et al. 1998. Trends Microbiol 6:66-71). The enzyme has been used to treat experimental aortic valve endocarditis caused by S. aureus (Patron, et al. 1999. Antimmicrob. Agents Chemother. 43:1754-5).

Engineering a lactobacillus to express GBS phage lysin is advantageous for several reasons. First, lactobacillus is the dominant commensal organism in the vagina. Second, it is also important that lactobacilli are considered very safe. Many foods, such as yogurt, buttermilk, sour cream, dried sausages, etc., contain high concentrations of live lactobacilli. In addition, commercially available lactobacillus preparations are now being used as probiotics and administered to people and cattle for their putative beneficial effects on gastrointestinal or urogenital health. Although their efficacy remains controversial, several lactobacillus preparations intended to colonize the intestine do appear to be beneficial and others at least do not cause any harm (Gorbach, S. L. 2000. Am. J. Gastroenterol. 95:S2-4). Interestingly, some strains of lactobacilli that were found to colonize the intestine were not able to persistently colonize the vagina (Gardiner, et al. 2002. Clin. Diagn. Lab. Inmunol. 9:92-6).

Several mouse models of GBS vaginal colonization have been described in the literature. For example, Medaglini, et al. (Medaglini, et al. Vaccine 15:1330-7) used the human oral commensal, Streptococcus gordonii, genetically modified to express a human papillomavirus protein, to vaginally immunize mice. They were able to establish a persistent vaginal colonization after a single inoculation with the organism. These authors subsequently reported that they were able to vaginally colonize mice with Lactobacillus casei (Medaglini, et al. 1998. Am. J. Reprod. Immunol. 39:199-208.). They expressed the E. coli heat labile toxin (LTB) in the lactobacillus using the broad-range conjugative transposon Tn916 (Medaglini, et al. 1998).

### METHODS FOR PROPHETIC EXAMPLE 1

Genetic Modification of Lactobacilli There is a significant amount of published information available on the genetic manipulation of lactobacilli. This is primarily because of the great economic value of the organisms in the dairy products industry. As discussed above, expression of GBS phage lysin is not harmful to the lactobacillus. In addition, it is not anticipated that susceptible bacteria, such as GBS, will not rapidly develop resistance to GBS phage lysin. As pointed out recently by Loeffler et al. (Loeffler, et al. 2001), the binding domains of phage lysins have probably evolved to target unique and essential molecules in the cell walls of their host species, making resistance to the enzymes very unlikely to develop.

Construction of an Expression-Secretion Cassette The assembly of the various elements required for efficient secretion of the GBS phage lysin or functional derivatives thereof by a lactobacillus will be carried out in several steps during construction of a specialized expression vector. This will be done using procedures similar to those described by Pouwels and coworkers (Pouwels, et al. 2001. Methods Enzymol. 336:369-89). The expression cassette will contain a strong constitutive promoter, a suitable translation initiation region, a signal peptide sequence, nucleic acid encoding for GBS phage lysin, or a functional derivative thereof, and a transcription termination sequence. Pouwels et al. (Pouwels, et al. 2001) used the strong promoter of the Lactobacillus casei L-lactate dehydrogenase (L-ldh) gene, a translation initiation region also from the L-ldh gene, the signal sequence of the proteinase gene, prtP, from L. casei, and the transcription terminator of the L. plantarum conjugate bile acid hydrolase gene (T_{cbh}). In an alternate embodiment, GBS phage lysin nucleic acid, or a functional derivative thereof may be integrated into the chromosome of the lactobacillus, as described below.

Construction of an Integration-Excision Vector The GBS phage lysin expression-secretion cassette will be inserted into the chromosome of a suitable lactobacillus strain using an integration strategy similar to that of Shimizu-Kadota (Shimizu-Kadota, 2001. J. Biotechnol. 89:73-79). This author constructed a special integration-excision vector for use in introducing foreign genes into the chromosome of a lactobacillus. The vector contained two copies of the selected gene, one N- and the other C-terminally truncated, located on either side of a lactobacillus phage attachment site (attP). The plasmid also contained a suitable integrase gene, an RNA primer region, a plasmid origin of replicon (ori), and an erythromycin resistance marker. The plasmid is electroporated into the lactobacillus where it inserts into the phage attachment site (attB) on the bacterial chromosome by site-specific recombination catalyzed by the integrase. Erythromycin-resistant integrants are then allowed to grow in the absence of antibiotic and erythromycin-sensitive colonies are isolated. These will result from homologous recombination within identical regions of the two copies of the GBS phage lysin gene cassette. The resulting recombinant will no longer have the antibiotic marker, the integrase, or the plasmid ori. It should then stably express DNA coding for GBS phage lysin, or a functional derivative thereof. Plasmid pMSK 742 is available and may be used directly, provided the lactobacillus we select possesses a suitable attB site. This should be readily apparent since chromosomal integration of plasmid electroporated into lactobacilli will result in erythromycin-resistant integrants. If a problem is encountered, an alternate strategy using the site-specific recombination system described by Alvarez et al. (Alvarez, et al. 1998. Virology 250:185-93) will be used. This system utilizes the attP site of lactobacillus phage A2 and has the potential advantage of being much less specific than the previous system. These authors cloned a 1.7 kb fragment of phage DNA from lysogenic Lactobacillus paracasei ATTC 27092 into an erythromycin resistant derivative of pUC18. The plasmid containing the recombination cassette, did not replicate in gram-positive bacteria, but did integrate into the chromosomes of all lactobacillus species tested. Regardless of which of the above site-specific integration systems is used, a special integration-excision vector will be constructed containing truncated copies of the GBS phage lysin gene, or a functional derivative thereof, arranged on either side of the attP site, following the strategy of Shimizu-Kadota (Shimizu-Kadota, 2001). A similar excision strategy has been used by the Applicants to remove integrated plasmid DNA during the construction of GBS hyaluronan lyase mutants using the shuttle/suicide vector pJL1055 (Li, et al. 1997. Proc. Natl. Acad. Sci. U S A 94:13251-6).

Bacterial Strains and Culture Conditions GBS strains from the Applicants'culture collection assembled over the past 25 years will be used. GBS will be grown in Todd-Hewitt broth, or on solid media composed of Trypticase Soy Agar (Becton Dickinson, Cockeysville, Md.) containing 5% sheep blood. Liquid media selective for GBS will be made by adding crystal violet (0.1 1μg/ml), nalidixic acid (15 μg/ml), and polymixin B sulfate (1 μg/ml) to Todd-Hewitt Broth (Gray, B. M. 1979. J. Clin. Microbiol. 9:466-470). Agar plates selective for GBS will be made by adding crystal violet (1 μg/ml), nalidixic acid (15 μg/ml), and polymixin B sulfate (10 μg/ml) to Trypticase Soy Agar. The Staphylococcus aureus strain used for routine CAMP testing will be stored frozen and freshly plated on blood agar plates at least weekly. Routine CAMP testing will be carried out on blood agar plates as previously described (Christie, et al. 1944. 22:197-200).

A large number of human vaginal lactobacillus strains are available to the Applicants. A total of 305 lactobacillus strains are available from ATCC, including one from a rodent strain (L. murinus). In initial experiments, a human vaginal lactobacillus isolate that produces H₂O₂ will be used. Peroxide production will be assessed using tetramethylbenzidine agar as described by previously (Eschenbach, et al. 1989. J. Clin. Microbiol. 27:251-6). Lactobacilli will be cultured in MRS liquid medium or on MRS agar plates under anaerobic conditions (DeMan, et al. 1960. J. Appl. Bact. 23:1305).

Vaginal Colonization of Mice Female IRC mice, approximately 13 weeks old, will be injected subcutaneously with 0.1 ml of 1 mg/ml estradiol valerate in castor oil. This solution is prepared by diluting 20 mg/ml estradiol valerate in castor oil (Delestrogen, Bristol-Myers Squibb Co., Princeton N.J.) with additional castor oil. One day later, prior to GBS inoculation, vaginal lavage will be carried out using a 0.2% solution of Triton X-100 in 0.9% NaCl. This is accomplished by rinsing the vaginal cavity 3 times with 50 μl of the lavage solution using a standard 200 μl conical plastic tip on a micropipettor. After 10 min, the vagina will be rinsed five times with 50 μl of 0.9% NaCl. One hour later the mice will be vaginally inoculated with 20 μl of a concentrated bacterial suspension (GBS, lactobacillus, or a mixture of both, ˜10⁸ cfu). The mice will then be vaginally swabbed every day for the next month using 0.6 mm ultrafine calcium alginate swabs (Fisher Scientific, Atlanta, Ga.). The swabs will be immediately placed into tubes containing 2 ml of selective medium for GBS and incubated at 37° C. for 24 hrs. Serial dilutions of these cultures will be spread on selective agar plates and incubated overnight. In our experience only GBS colonies are obtained. However, the colonies will be picked, CAMP tested on sheep blood agar plates, and also tested for hippuricase activity, to confirm that they are GBS. On occasion, nitrous acid extracts of selected colonies will be immunologically tested using our specific monoclonal antibodies for the presence of the group B-specific polysaccharide and the serotype-specific polysaccharide in order to further verify their identity. This approach of using selective medium to detect vaginal colonization by GBS is widely used in medicine today and is specifically recommended by CDC guidelines (Centers for Disease Control and Prevention. 1999. Morbid. Mortal. Weekly Rep. 48:426-428). In addition, a real-time PCR procedure for rapidly assessing the extent of vaginal GBS colonization will be employed.

Vaginal colonization of mice with a human vaginal lactobacillus isolate will also be accomplished. Medaglini et al. previously reported vaginally colonizing mice with a human strain of Lactobacillus casei (Medaglini, et al. 1998). These workers intentionally inoculated mice at the estrous stage of their reproductive cycles in order to obtain efficient bacterial colonization. We will use estrogen-treated mice as described above in order to achieve long-term colonization with GBS. However, it is possible that this hormone-treatment may also help facilitate persistent vaginal colonization with lactobacilli.

Lactobacillus vaginal colonization will be routinely assessed using a semi-quantitative culture procedure routinely used for human specimens (Krohn, et al. 1991. J. Infect. Dis. 164:88-93). Vaginal swabs from mice will be placed into 1.5 ml of pre-reduced balanced salt solution and serial 1:10 dilutions will be plated on MRS agar. Plates will be incubated in an anaerobic jar for 3 days at 37° C. Colonies will be enumerated and lactobacilli will be identified by colony morphology, gram staining, a negative catalase test, and by gas chromatography (Hillier, et al.. 1995. p. 587-602. In P. R. Murray, E. Baron, M. A. Pfaller, F. C. Tenover, and R. H. Yolken (ed.), Manual of Clinical Microbiology., Washington D.C.). The gas chromatographic method measures 12 different organic acids produced by various bacteria in order to positively identify lactobacilli (Suttler, et al. 1986. p. 71-78, Wadsworth Anaerobic Bacteriology Manual. Star Publishing Co., Belmont, Calif.).

Effect on GBS Persistence of Co-Colonization with a GBS Phage Lysin-Secreting Lactobacillus. Two groups of mice each will be co-colonized with GBS and a lactobacillus. The lactobacillus genetically modified to secrete GBS phage lysin will be used in the test group and the parental lactobacillus strain in the control group. Mice will be vaginally swabbed daily and GBS colonization status determined. Since the initial experiments will use a plasmid expression-secretion vector for the GBS phage lysin gene, it will be necessary to confirm that the lactobacillus has not lost the plasmid during carriage in the mouse. We will therefore verify that lactobacillus colonies recovered from the vaginal swabs continue to express their antibiotic markers. We then will engineer a lactobacillus to express GBS phage lysin by integrating the GBS phage lysin gene directly into the bacterial chromosome. We will then confirm that lactobacilli recovered on vaginal swabs continue to secrete the enzyme. The ability of lactobacillus engineered to secrete GBS phage lysin to prevent GBS from establishing persistent colonization will be determined.

In addition, the ability of a lactobacillus to eliminate an established GBS infection will also be determined. This will again be done using two groups of mice. After both groups have been colonized with GBS for one week, the test group will be vaginally inoculated with 10⁸ cfu of the engineered lactobacillus and the control group with the same dose of the unmodified parental lactobacillus strain. Persistence of GBS in the two groups will be determined as before.

What is claimed:

1. An isolated nucleic acid comprising the nucleotide sequence of SEQ ID NO: 1 or the complement of SEQ ID NO: 1.

2. The nucleic acid of claim 1 comprising a functional derivative of the nucleotide sequence of SEQ ID. NO: 1.

3. The nucleic acid of claim 2 where the functional derivative is selected from the group consisting of a fragment, and a degenerate variant.

4. The nucleic acid of claim 3 where the functional derivative is a fragment and the fragment comprises a contiguous sequence of at least 15 nucleotides of SEQ ID NO: 1 or the complement of SEQ ID NO: 1.

5. The nucleic acid of claim 3 where the functional derivative is a fragment and the fragment comprises a contiguous sequence of at least 20 nucleotides of SEQ ID NO: 1 or the complement of SEQ ID NO: 1.

6. The nucleic acid of claim 1 comprising a nucleotide sequence which is at least 50% identical to the nucleotide sequence of SEQ ID NO: 1.

7. The nucleic acid of claim 1 comprising a nucleotide sequence which is at least 80% identical to the nucleotide sequence of SEQ ID NO: 1.

8. The nucleic acid of claim 1 comprising a nucleotide sequence which is at least 90% identical to the nucleotide sequence of SEQ ID NO: 1.

9. The nucleic acid of claim 1 comprising a nucleotide sequence which is at least 95% identical to the nucleotide sequence of SEQ ID NO: 1.

10. The nucleic acid of claim 1 where said nucleotide sequence encodes a polypeptide having the amino acid sequence of SEQ ID NO: 2, a fragment of SEQ ID NO: 2 at least 5 amino acid residue in length, or a degenerate variant of SEQ ID NO: 2.

11. The nucleic acid of claim 1 where said nucleotide sequence encodes a polypeptide having the amino acid sequence of SEQ ID NO: 2 wherein (a) at position 26 the amino acid cysteine is replaced by serine; (b) at position 44 the amino acid cysteine is replaced by serine; or (c) both (a) and (b).

12. The nucleic acid of claim 1 where said nucleotide sequence encodes a polypeptide the amino acid sequence of which is at least 80% identical to SEQ ID NO: 2.

13. An isolated nucleic acid comprising a sequence that hybridizes under highly stringent conditions to a hybridization probe the nucleotide sequence of which consists of SEQ ID NO: 1, the complement of SEQ ID NO: 1, a fragment of SEQ ID NO: 1 at least 15 nucleotides in length or the complement of a fragment of SEQ ID NO: 1 at least 15 nucleotides in length.

14. A purified polypeptide the amino acid sequence of which comprises SEQ ID NO: 2, or a degenerate variant of SEQ ID NO: 2.

15. The purified polypeptide of claim 14 the amino acid sequence of which comprises a fragment of SEQ ID NO: 2 of at least 5 consecutive amino acids.

16. The purified polypeptide of claim 14 the amino acid sequence of which comprises a fragment of SEQ ID NO: 2 of at least 5 consecutive amino acids, wherein said polypeptide is immunologically reactive with an anti-GBS phage lysin antibody.

17. The purified polypeptide of claim 14 the amino acid sequence of which is at least 50% identical to SEQ ID NO: 2.

18. The purified polypeptide of claim 14 the amino acid sequence of which is at least 80% identical to SEQ ID NO: 2.

19. The purified polypeptide of claim 14 the amino acid sequence of which is at least 90% identical to SEQ ID NO: 2.

20. The purified polypeptide of claim 14 the amino acid sequence of which is at least 95% identical to SEQ ID NO: 2.

21. The purified polypeptide of claim 14 the amino acid sequence of which comprises a fragment of SEQ ID NO: 2 selected from the group consisting of residues 1-107, 6-107, 6-443, 1-344, 145-344, 145-443 and 6-344.

22. The purified polypeptide of claim 21 where the polypeptide express at least one of an endopeptidase or a glycosidase activity.

23. The purified polypeptide of claim 14 wherein (a) at position 26 the amino acid cysteine is replaced by serine; (b) at position 44 the amino acid cysteine is replaced by serine; or (c) both (a) and (b).

24. The purified polypeptide of claim 23 where the polypeptide does not express an endopeptidase activity.

25. The purified polypeptide of claim 14 where the polypeptide express an endopeptidase and a glycosidase activity.

26. An expression vector comprising the nucleic acid of claim 1 operably linked to an expression control sequence.

27. A non-human host cell comprising the expression vector of claim 26.

28. A method of producing a GBS phage lysin, the method comprising culturing the host cell of claim 27 under conditions permitting expression of the GBS phage lysin from the expression vector.

29. The method of claim 28 further comprising purifying the GBS phage lysin.

30. A method of treating or preventing a condition caused by a bacteria in an individual in need of such treatment or prevention, the method comprising the step of administering to the individual an effective amount of a therapeutic agent, the therapeutic agent comprising the polypeptide of claim 14 in a pharmaceutically acceptable carrier, said bacteria having a peptidoglycan structure comprising an interpeptide bridge consisting of (L-Ala)₂ or L-Ala-L-Ser.

31. The method of claim 30 where the bacteria is a group B streptococci and the condition is a vaginal colonization of the group B streptococci.

32. The method of claim 31 where the pharmaceutically acceptable carrier is a tampon, pad or a douche.

33. The method of claim 30 where the bacteria are selected from the group consisting of group A streptococci, group B streptococci, group C streptococci, group E streptococci, and group G streptococci.

34. The method claim 30 further comprising the step of administration of a modulating compound in a pharmaceutically acceptable carrier.

35. A method of disrupting the cell walls of a bacteria comprising the step of contacting the bacteria with the protein of claim 14, said bacteria having a peptidoglycan structure comprising an interpeptide bridge comprising (L-Ala)₂ or L-Ala-L-Ser.

36. The method of claim 35 where the bacteria are selected from the group consisting of group A streptococci, group B streptococci, group C streptococci, group E streptococci, and group G streptococci.


SEQUENCE LISTING (5 sequences)

SEQ ID NO: 1 - DNA, 1329 bp, Streptococcus agalactiae bacteriophage B30
atggcaacttatcaagaatataaaagcaggtcaaatggtaacgcttatgatattgatgggtctttcggtgcacaatgttgggatggctacgcagattactgtaagtatctaggactgccatacgcaaactgtacaaatacaggatacgcaagggatatatgggagcaacgtcacgaaaatggtatcttaaactattttgatgaagtggaagttatgcaagctggtgatgttgctatttttatggttgttgacggtgtaacgccttacagtcatgtagcaatttttgacagcgatgcaggaggcggatatggctggtttttggggcaaaatcaagggggtgctaacggtgcatacaatcttgtaaaaatcccatattcagcaacttatccaacagcctttagaccaaaaagctttaaaaacgctgttactgtaactgataataccggtttaaataaaggtgattactttatcgatgtatcggcttatcaacaagcagatttaacaacgacttgtcagcaggcgggcactacaaaaacgattatcaaggtatccgagtcaattgcttggctgtctgacagacatcaacaacaagcaaacacaagcgacccaattggctattaccactttggacgttttggcggtgatagtgctttagcgcaacgggaagcagacttatttttgtctaacttaccaagcaaaaaagtatcatacttagtcattgactatgaagattccgcaagcgcagacaagcaagctaacacaaatgcagttattgcatttatggataaaatcgctagcgctggatataagcctatttattacagctataaaccatttacgcttaataatattgattatcagaaaattatcgctaagtaccctaatagcatttggatagctggttatccagactacgaagtacgaacagagccgctgtgggagttcttcccttcaatggatggtgttcgctggtggcagtttacaagtgtaggagtagcaggtggtttagataaaaatattgtgttgttagcagatgatagtagcaaaatggatatacctaaggttgataagccacaagaacttactttttatcaaaaactagctactaacactaaattagacaactcaaatgtaccttattacgaagcaactcttagcacagactattatgtagagtctaagccaaacgcaagtagcgctgataaagaatttatcaaggcaggaactcgtgtaagagtttatgaaaaagtgaatggatggtcacgcattaaccatccagagtcggcgcaatgggtagaagataactacttagttaacgcaacagatatg

SEQ ID NO: 2 - protein, 443 aa, Streptococcus agalactiae bacteriophage B30
  ref <308>: DATABASE ACCESSION NUMBER: GenBank/AY149214
  ref <309>: DATABASE ENTRY DATE: 2003-06-26
  ref <313>: RELEVANT RESIDUES: (1)..(443)
MATYQEYKSRSNGNAYDIDGSFGAQCWDGYADYCKYLGLPYANCTNTGYARDIWEQRHENGILNYFDEVEVMQAGDVAIFMVVDGVTPYSHVAIFDSDAGGGYGWFLGQNQGGANGAYNLVKIPYSATYPTAFRPKSFKNAVTVTDNTGLNKGDYFIDVSAYQQADLTTTCQQAGTTKTIIKVSESIAWLSDRHQQQANTSDPIGYYHFGRFGGDSALAQREADLFLSNLPSKKVSYLVIDYEDSASADKQANTNAVIAFMDKIASAGYKPIYYSYKPFTLNNIDYQKIIAKYPNSIWIAGYPDYEVRTEPLWEFFPSMDGVRWWQFTSVGVAGGLDKNIVLLADDSSKMDIPKVDKPQELTFYQKLATNTKLDNSNVPYYEATLSTDYYVESKPNASSADKEFIKAGTRVRVYEKVNGWSRINHPESAQWVEDNYLVNATDM

SEQ ID NO: 3 - DNA, 1880 bp, Streptococcus agalactiae bacteriophage B30
  stem_loop (66)..(99)
  stem_loop (1512)..(1539)
  ref <308>: DATABASE ACCESSION NUMBER: GenBank/AY149214
  ref <309>: DATABASE ENTRY DATE: 2003-06-26
  ref <313>: RELEVANT RESIDUES: (1)..(1880)
ccacgagccaaaaaaataggaggggacatgcgagcaatcactaaaatagcaatggtactagcgatagcaatcctgtacataccgcttgcagtgattgctttttttatttatccgatttatttcctttttggaaaggaggagtaaatggcaacttatcaagaatataaaagcaggtcaaatggtaacgcttatgatattgatgggtctttcggtgcacaatgttgggatggctacgcagattactgtaagtatctaggactgccatacgcaaactgtacaaatacaggatacgcaagggatatatgggagcaacgtcacgaaaatggtatcttaaactattttgatgaagtggaagttatgcaagctggtgatgttgctatttttatggttgttgacggtgtaacgccttacagtcatgtagcaatttttgacagcgatgcaggaggcggatatggctggtttttggggcaaaatcaagggggtgctaacggtgcatacaatcttgtaaaaatcccatattcagcaacttatccaacagcctttagaccaaaaagctttaaaaacgctgttactgtaactgataataccggtttaaataaaggtgattactttatcgatgtatcggcttatcaacaagcagatttaacaacgacttgtcagcaggcgggcactacaaaaacgattatcaaggtatccgagtcaattgcttggctgtctgacagacatcaacaacaagcaaacacaagcgacccaattggctattaccactttggacgttttggcggtgatagtgctttagcgcaacgggaagcagacttatttttgtctaacttaccaagcaaaaaagtatcatacttagtcattgactatgaagattccgcaagcgcagacaagcaagctaacacaaatgcagttattgcatttatggataaaatcgctagcgctggatataagcctatttattacagctataaaccatttacgcttaataatattgattatcagaaaattatcgctaagtaccctaatagcatttggatagctggttatccagactacgaagtacgaacagagccgctgtgggagttcttcccttcaatggatggtgttcgctggtggcagtttacaagtgtaggagtagcaggtggtttagataaaaatattgtgttgttagcagatgatagtagcaaaatggatatacctaaggttgataagccacaagaacttactttttatcaaaaactagctactaacactaaattagacaactcaaatgtaccttattacgaagcaactcttagcacagactattatgtagagtctaagccaaacgcaagtagcgctgataaagaatttatcaaggcaggaactcgtgtaagagtttatgaaaaagtgaatggatggtcacgcattaaccatccagagtcggcgcaatgggtagaagataactacttagttaacgcaacagatatgtaaaaataaacgaaaggaaaagctcctttagataagacaaatgccctcgctttgcgggggctatttttattgaaatattgaaatctctttataaaaatagtaaaatagtttcgctattataaagaaagttgttatcaatgaataatctagttcttcctcagaatttaaacaaatataacattacgaaaatcgttaccaatttcaatagattacttgctttaagtgataacaggacacttacagtagatatgagaaacattgagtttgcggaacctagtggagtaatttcgttatataatatgttaacttttgctacaaaaagaaaagatgcaaacatcaagtggttaatatgcgaagaaagctctttaaataaacgtcaaaggcaagctatgttgtatctagtagat

SEQ ID NO: 4 - DNA, 35 bp, Artificial
  PCR Primer
gcactacatatggcaacttatcaagaatataaaag

SEQ ID NO: 5 - DNA, 38 bp, artificial
  PCR Primer
gtggtgctcgagcatatctgttgcgttaactaagtagc